(19) United States
(12) Patent Application Publication
(10) Pub. No.: US 2025/0000638 A1
(43) Pub. Date: Jan. 2, 2025

(54) SILICONE-BASED IMPLANTS WITH EARLY PREFERENTIAL POLARIZATION TOWARDS AN M2 PHENOTYPE POST-IMPLANTATION

(71) Applicant: DiFusion, Inc., Austin, TX (US)

(72) Inventors: Derrick Johns, Liberty Hill, TX (US); Joseph J. Crudden, Hudson, NH (US)

(21) Appl. No.: 18/753,329

(22) Filed: Jun. 25, 2024

Related U.S. Application Data
(60) Provisional application No. 63/523,672, filed on Jun. 28, 2023.

(51) Int. Cl. A61F 2/12; A61L 27/10; A61L 27/44; A61L 27/50; C08K 3/34

(52) U.S. Cl. CPC A61F 2/12; A61L 27/10; A61L 27/446; A61L 27/505; C08K 3/34; C08K 2003/343

(57) ABSTRACT

Breast prostheses that are anti-biofilm implantable biomaterial devices that optionally can elute therapeutic ions such as magnesium, silver, copper and/or zinc. In certain embodiments, the devices are hydrophilic and include a capsular contracture reducing or inhibiting agent.

This application claims priority of U.S. Provisional Application Ser. No. 63/523,672 filed Jun. 28, 2023, the disclosure of which is incorporated herein by reference in its entirety.

### BACKGROUND

Biomaterials may be surgically implanted into the body for various reasons, including orthopedic applications (e.g., hip replacement, skull flaps, dental implants, spinal procedures, knee replacement, bone fracture repair, etc.), surgical repair applications (e.g., ACL screws, surgical meshes, etc.), breast implantation, and others. In view of the structural integrity required by many such devices, particularly those involving bone repair or replacement, and biocompatibility requirements, materials of fabrication are limited and generally consist of metal, plastic and composites. Each has its advantages and disadvantages.

Suitable polymeric materials for implant applications should interact well with tissue, and also should be recognized by the host as natural so as to minimize or avoid becoming encapsulated by a fibrous apposition layer of soft tissue. The role of the neutralization of infectious agents, and the host response to foreign materials such as surgical implants in normal tissue/organ development and tissue regeneration, is important. Immune cells such as neutrophils, macrophages, and lymphocytes possess robust plasticity with respect to phenotype. For example, macrophages typically show a marked pro-inflammatory (M1-like) phenotype when presented with certain antigens (e.g., synthetic foreign materials or bacteria), but then transition to pro-healing, anti-inflammatory and constructive phenotype (M2-like) when subsequently influenced by alternative signaling molecules. A “normal” response to injury involves an initial pro-inflammatory cell response that must then transition to a pro-healing phenotype, lest there be continuous, non-healing inflammation and tissue destruction. The phenotype of cells such as macrophages can be determined, at least in part, by the expression of certain markers that are detected by immunolabeling. Macrophage phenotype during the early response (i.e., 7-14 days) to an implanted foreign material is predictive of the downstream outcome. An early M1-like response has been associated with chronic inflammation and fibrosis; whereas an early M2-like response has been associated with minimal fibrosis and constructive and functional tissue remodeling.

It is therefore important that implantable biomaterials be developed that promote activation of one or more genes associated with an M2-like macrophage phenotype. A desirable M1/M2 macrophage phenotype balance, and in particular, the early preferential polarization towards an M2 phenotype after implantation, can lead to a shorter pro-inflammatory period and earlier reparative process, which can be critical for effective tissue integration and ultimately implant success.

This is particularly true for silicone-based (e.g., silicone elastomers) implants such as implantable breast prostheses and tissue expanders, such as those used for reconstruction (e.g., following mastectomy), augmentation, cosmetics purposes, etc. Indeed, when inserted into a host, such implants are recognized as a foreign body by the immune system of the host, resulting in deleterious fibrosis and/or encapsulation. This can result in one or more of distortion of the implant, displacement of the implant, implant palpability, pain or discomfort, scar tissue formation, capsular contracture and other issues that negatively impact the host. Such post-surgical complications can be so severe that further surgical intervention is required, often multiple times.

Although the formation of a capsule of scar tissue around a breast implant has certain advantages, including assisting the implant in remaining in place, it also can lead to aesthetic issues and discomfort for the host, and can even lead to implant shell rupture. Capsular contracture is so pervasive that its severity is rated using a grading system (Baker classification) based on clinical examination and/or imaging. The rating system includes Grade I, where the host is asymptomatic with the breast being soft and having a normal appearance; Grade II where the host exhibits minor cosmetic issues; Grade III where the host exhibits obvious cosmetic issues; and Grade IV where the breast has hardened, appears abnormal, and induces pain in the host. Grades III and IV are clinically significant. It is generally agreed in the medical community that the etiology of capsular contracture includes an inflammatory component.

Accordingly, the promotion of activation of one or more genes associated with an M2-like macrophage phenotype, and a desirable M1/M2 macrophage phenotype balance is of particular importance where the implant is a breast prostheses, as chronic inflammation and concomitant fibrosis and implant encapsulation are well-documented problems with such devices.

In addition, as microorganisms come in close proximity to the surface of the device, they will either be attracted or repelled by it depending on the sum of the different non-specific interactions. In biological systems, hydrophobic/hydrophilic interactions play an important role in the pathogenesis of a wide range of microbial infections. Many implant materials are hydrophobic materials and bacteria tend to adhere easily to these types of surfaces. They are also organic materials which do not carry significant surface charges. Consequently, it would be desirable to develop a breast prosthesis that has reduced hydrophobic properties, and/or that has a net negative charge, particularly at an exposed surface when implanted into a host.

Accordingly, it is an object of embodiments disclosed herein to reduce capsular contracture in a host patient having one or more implanted breast prostheses or tissue expanders.

It is a further object of embodiments disclosed herein to reduce capsular contracture in a host patient having one or more implanted breast prostheses or tissue expanders to achieve a Baker classification of Grade III or less, preferably Grade II or less.

It is a further object of embodiments disclosed herein to reduce deformity around an implanted breast prosthesis or tissue expander as a result of, for example, capsular contracture.

It is a still further object of embodiments disclosed herein to reduce or eliminate pain or discomfort arising as a result of capsule contracture in a host having one or more implanted breast prostheses or tissue expanders.

Accordingly, embodiments disclosed herein relate to implantable medical devices such as breast prostheses that are composed of, and/or are coated with, and/or contain, one or more silicone elastomers, wherein the elastomers include a capsular contracture reducing or inhibiting agent, wherein the agent is a ceramic such as an aluminosilicate, preferably a zeolite.

The ceramic present in the device may optionally be loaded with one or more ion-exchangeable cations. The one or more cations may elute from the device upon exposure to bodily fluid, and provide a therapeutic effect to the host, such as antimicrobial and/or anticoagulative activity.

### SUMMARY

The shortcomings of the prior art have been overcome by embodiments disclosed herein, which include engineered implantable biomaterial devices that optionally can elute therapeutic ions. In certain embodiments, the implants are soft tissue implants such as breast prostheses and are so shaped or configured. In certain embodiments, the implants are for cosmetic applications, including for reconstructive surgery and augmentation. In certain embodiments, the breast prostheses are implantable into a host and maintain a desired cosmetic shape over time when so implanted. In some embodiments, the breast prostheses are implantable into a living host in direct contact with breast tissue of the host. In certain embodiments, the breast prostheses are non-loading bearing. Suitable hosts include mammals, particularly humans. In some embodiments, the devices are composed of, contain or are coated with a medical grade elastomer such as a silicone elastomer. In some embodiments, the elastomer includes a ceramic material, preferably a zeolite, and the ceramic material optionally may be loaded with one or more therapeutic metal ions, such as silver, copper and/or zinc, that exhibit a therapeutic effect such as antimicrobial or anticoagulative properties when implanted into a body and exposed to bodily fluid or tissue. The devices, when implanted into a body and exposed to bodily fluid, may elute metal ions in a therapeutically effective amount.

In certain embodiments, the breast prostheses is positioned under the pectoral or pectoralis major muscle of a living host e.g., submuscular pocket placement). In certain embodiments, the breast prostheses is positioned directly behind the breast tissue, over the pectoral muscle (e.g., subglandular or subfascial pocket implantation). In certain embodiments, the breast prosthesis is positioned partially under the pectoralis major muscle (e.g., dual plane implantation).

In certain embodiments, disclosed are methods of imparting therapeutic activity to devices by controlling the delivery of certain cations through ion-exchange via a ceramic material, preferably zeolite, incorporated in the device introduced in a patient. In some embodiments, the ceramic material does not contain a metal ion, yet reduces or inhibits capsular contracture. In some embodiments, the ceramic material imparts hydrophilicity and a negative charge to the device. The presence of the ceramic material helps prevent biofilm formation, fibrosis and/or encapsulation. In embodiments where antimicrobial metal ions are present, the elastomer/zeolite combination increases the ability of the therapeutic moieties to permeate in and kill the bacterial pathogen.

In certain embodiments the breast prostheses are tissue expanders. Tissue expanders are typically utilized in surgical breast reconstruction following a mastectomy in order to rebuild injured or deformed breasts, or as part of gender reassignment surgery. For example, after mastectomy, a collapsed or partially inflated tissue expander may be placed under or over the pectoralis major muscle behind the area of the removed breast to create a new breast pocket for subsequent insertion of a breast implant or the host's own tissue. The tissue expander may be post-operatively filled (e.g., inflated) with a filler material, such as saline or air, over several days, weeks, or even months until the breast pocket achieves a desired volume and shape to accommodate the breast implant which ultimately may replace the expander. The tissue expander can be used, for example, to stretch skin remaining from a mastectomy to a larger dimension, particularly to a dimension that can accommodate a breast implant.

In some embodiments, the aluminosilicate is represented by the formula XM_{2/n}O·Al₂O₃·YSiO₂·ZH₂O wherein M represents an ion-exchangeable ion, n represents the atomic valency of the (metal) ion, X and Y represent coefficients of metal oxide and silica respectively, and Z represents the number of water of crystallization.

The ceramic material may be incorporated into the silicone elastomer formulation to create a composite material with ceramic character that confers charge to the surface and renders it hydrophilic. In addition, the ceramic material can provide ion-exchange sites which optionally can be loaded with one or more therapeutic metal ions that elute when in contact with the bodily fluid or tissue of a host, thereby imparting therapeutic activity to the implant site, such as antimicrobial activity and/or anti-coagulating activity. However, poorly controlled release of metal ions because of device design can result in the deleterious accumulation of excess metal ions (e.g., silver, copper) in the host over time. Carefully controlled release of therapeutic metal ions, from ion exchange ceramics such as zeolites, materials which are used to fabricate medical devices can provide for precision controlled release of the correct, safe and efficacious level of therapeutic ion(s).

### DETAILED DESCRIPTION

Although specific terms are used in the following description for the sake of clarity, these terms are intended to refer only to the particular structure of the embodiments selected for illustration in the drawings. In the drawings and the following description below, it is to be understood that like numeric designations refer to components of like function.

The singular forms “a,” “an,” and “the” include plural referents unless the context clearly dictates otherwise.

As used in the specification, various devices and parts may be described as “comprising” other components. The terms “comprise(s),” “include(s),” “having,” “has,” “can,” “contain(s),” and variants thereof, as used herein, are intended to be open-ended transitional phrases, terms, or words that do not preclude the possibility of additional components.

All ranges disclosed herein are inclusive of the recited endpoint and independently combinable (for example, the range of “from 2% to 10” is inclusive of the endpoints, 2% and 10%, and all the intermediate values).

As used herein, approximating language may be applied to modify any quantitative representation that may vary without resulting in a change in the basic function to which it is related. Accordingly, a value modified by a term or terms, such as “about” and “substantially,” may not be limited to the precise value specified, in some cases. The modifier “about” should also be considered as disclosing the range defined by the absolute values of the two endpoints. For example, the expression “from about 2 to about 4” also discloses the range “from 2 to 4.”

It should be noted that some terms used herein are relative terms. For example, the terms “upper” and “lower” are relative to each other in location, i.e. an upper component is located at a higher elevation than a lower component, and should not be construed as requiring a particular orientation or location of the structure. As a further example, the terms “interior”, “exterior”, “inward”, and “outward” are relative to a center, and should not be construed as requiring a particular orientation or location of the structure.

The terms “top” and “bottom” are relative to an absolute reference, i.e. the surface of the earth. Put another way, a top location is always located at a higher elevation than a bottom location, toward the surface of the earth.

The terms “medical device”, “device”, “apparatus”, “implant”, “medical implant”, “prosthesis”, “prostheses” and the like may be used herein synonymously and refer to any object or objects configured to be positioned partially of wholly in the body of a host patient, such as a mammal, for one or more therapeutic, prophylactic or cosmetic purposes, including but not limited to tissue augmentation, contouring, void filling, and/or repairing or restoring damaged tissues. The terms “breast prosthesis” or “breast prostheses” include a breast implant or implants and a tissue expander or expanders. In certain embodiments, the breast prostheses are anatomically shaped in the form of a breast.

Certain embodiments relate to a biomaterial useful as a breast prosthesis comprising a base material such as a silicone elastomer. Various embodiments relate to breast prostheses comprising silicone and a capsular contracture reducing or inhibiting agent. A “reducing or inhibiting agent” as used herein includes an agent that results in a statistically significant decrease (relative to what would be expected to occur in the absence of the agent) in capsular contracture and/or a statistically significant improvement (relative to what would be expected to occur in the absence of the agent) in the inflammatory response of the host to the implantation such as by encouraging a rapid transition from M1 proinflammatory macrophage phenotype to the M2 macrophage and thus preferential polarization towards an M2 phenotype after implantation. Improved implant function (e.g., enhanced appearance), less scar tissue formation and/or a reduction in the need for repeat or repeated surgical intervention may result.

In various embodiments, the capsular contracture reducing or inhibiting agent comprises or consists essentially of a ceramic material or aluminosilicate. In various embodiments, it comprises or consists essentially of a zeolite.

As used herein, the phrase “consisting essentially of” limits the scope of a claim to the specified materials or steps and those that do not materially affect the basic and novel characteristics of the claimed subject matter. The term permits the inclusion of elements or steps which do not materially affect the basic and novel characteristics of the apparatus, system or method under consideration. Accordingly, the expressions “consists essentially of” or “consisting essentially of” mean that the recited embodiment, feature, component, step, etc. must be present and that other embodiments, features, components, steps, etc., may be present provided the presence thereof does not materially affect the performance, character or effect of the recited embodiment, feature, component, step, etc. The presence of an operation or step that has no material effect on the sample or product is permitted. For example, a capsular contracture reducing or inhibiting agent consisting essentially of a zeolite excludes other components or ingredients in the agent that would materially change the agent or the prosthesis.

In certain embodiments, the silicone is or comprises vulcanized silicone, which has high tensile strength and is resistance to tearing. In certain embodiments the silicone is or comprises poly (dimethyl siloxane) or polydiphenylsiloxane elastomer. In certain embodiments, fumed amorphous silica may be added to the elastomer.

In certain embodiments, the breast implant or tissue expander is three-dimensional and has a flexible silicone elastomeric shell, wherein the shell is fillable (e.g., inflatable) with or is filled (e.g., inflated) with a liquid or a gel, such as intraoperatively, via one or more valves. In some embodiments, the shell defines an implant volume, some or all of which may be fillable or filled. Suitable fluids containable or contained in the shell include those conventionally used with breast implants, such as saline (e.g., sterile isotonic saline consistent with United States Pharmacopeia (USP) standards for Normal Physiological Saline (injection grade), which has a concentration of 0.15 M and a pH of 7.2 to 7.4). Suitable gels containable or contained in the shell include those conventionally used with breast implants or tissue expanders, such as silicone gel, including poly (dimethyl siloxane). The shell may be filled prior to, during and/or after implantation into a host. In certain embodiments the implant may have a fixed volume, such as a fixed volume single lumen silicone gel-filled or saline-filled implant). In certain embodiments the implant may have an adjustable volume, such as an adjustable volume multiple lumen silicone gel-filled or saline-filled implant. In certain embodiments, the implant comprises multiple layers of silicone elastomer. In certain embodiments, a desired or predetermined thickness of the implant is achieved by the addition of layers of silicone elastomer. In certain embodiments the implant is biologically compatible and non-absorbable by the host.

Regardless of whether the prosthesis is a single layer or multiple layer device, the capsular contracture reducing or inhibiting agent preferably is incorporated into at least the outer layer of the device that is exposed to bodily fluid or tissue once implanted in a host. In certain embodiments, the capsular contracture reducing or inhibiting agent is present in multiple layers of the device.

The breast prostheses in accordance with embodiments disclosed herein may be made by methods typical for silicone based implants devoid of a capsular contracture reducing or inhibiting agent, with one or more additional steps to incorporate the agent. For example, they may be manufactured by dipping an implant-shaped template or mandrel into a silicone solution to uniformly coat the template. The template is then placed in a hot, laminar flow cabinet or the like to allow for the polymer to cure around the template. This process can be repeated a plurality of times in order to increase the thickness of the implant; i.e., to form multiple layers. In certain embodiments, the ceramic material may be mixed into the elastomer at the desired levels prior to elastomer curing.

In some embodiments, the device has a main body region and an exposed surface region, the exposed surface region being configured to be exposed to bodily fluid and/or tissue of a host when the device is implanted in the host. Preferably the capsular contracture reducing or inhibiting agent is present in at least a portion of the exposed surface region.

In some embodiments, the biomaterial may have a capsular contracture reducing or inhibiting agent such as a ceramic material incorporated in the elastomer and/or on the surface of the elastomer. The presence of the agent imparts hydrophilicity and a negative charge to the device, and provides available ion-exchange sites for the optional incorporation of metal ions that can be eluted into the host after implantation. Thus, the capsular contracture reducing or inhibiting agent optionally may be loaded with one or more therapeutic metal ions that exhibit therapeutic properties when implanted into a body and exposed to bodily fluid or tissue. Suitable ions include silver, copper, zinc, mercury, tin, magnesium, lead, gold, bismuth, cadmium, chromium, strontium and thallium ions, calcium, silicon or combinations of one or more of the foregoing. Such devices, when implanted into a body and exposed to bodily fluid, may elute metal ions in a therapeutically or prophylactically effective amount. In certain embodiments, the source of therapeutic or prophylactic activity includes ion-exchangeable cations contained in a zeolite. The metal ions may include one or more divalent cations that contribute to integrin-ligand binding affinity.

Suitable amounts of ceramic material such as zeolite particles incorporated in a prosthesis elastomer range from 0.01 to as much as 95 wt. %. In certain embodiments, the surface of the prosthesis comprises ceramic material such as zeolite in an amount from about 8% to about 12 wt. %. In certain embodiments, the amount is greater than 12% by weight. In certain embodiments, the surface comprises ceramic material such as zeolite in an amount equal to or greater than 15% by weight. In certain embodiments, the surface comprises ceramic material such as zeolite in an amount equal to or greater than 20% by weight. In certain embodiments, the surface comprises ceramic material such as zeolite in an amount equal to or greater than 25% by weight. In certain embodiments, the surface comprises ceramic material such as zeolite in an amount equal to or greater than 50% by weight. In certain embodiments, the surface region comprises ceramic material such as zeolite in an amount equal to or greater than 75% by weight. In certain embodiments, the surface region comprises ceramic material such as zeolite in an amount as high as 95% by weight. The method used to coat or otherwise incorporate the ceramic into the elastomer is not particularly limited, and can include mixing the particles into the viscous elastomer liquid. Spraying, painting or dipping also may be used. The ceramic species can be a surface coating, can be incorporated or embedded into the elastomer (e.g., after curing), or can be both a surface coating and incorporated or embedded into the elastomer. Thus, unlike prior art implants that can a releasable agent that is said to reduce inflammation and fibrous encapsulation, embodiments disclosed herein relate to a capsular contracture reducing or inhibiting agent that may be permanently part of the implant itself and is not releasable or released therefrom. Where the implant is comprised of multiple layers of elastomer, the capsular contracture reducing or inhibiting may be incorporated into at least the outer layer of the implant. Alternatively, it may be incorporated into all of the layers of the implant. Alternatively still, it may be incorporate into one or more layers of the implant. The amount of the capsular contracture reducing or inhibiting agent need not be the same in each layer; a gradient of capsular contracture reducing or inhibiting agent in the implant may be used. In a preferred embodiment, it is incorporated into at least the outer layer of the implant such that it is available for contact with bodily fluids and/or tissue of the host once implanted. The zeolite changes the surface topography, charging characteristics, and pH of the resulting composite in a predictable, suitable manner for the surgical environment and long-term healing of the host patient into which the device is implanted. Attributes imparted by the zeolite include biocompatibility, negative charge, hydrophilicity, preferential polarization towards an M2 phenotype post-implantation, and promotion of cell adhesion. Attributes that may be provided by the base elastomer include radiolucency, biocompatibility, durability and versatility. The resulting composite blend provides a uniform material construct and excellent workability.

The hydrophilicity imparted by the ceramic material such as zeolite results in an engineered biomaterial that interacts with the tissue of the patient and induces fusion. The presence of the ceramic material such as zeolite also results in a rapid transition from Ml proinflammatory macrophage phenotype to the M2 macrophage phenotype, thereby minimizing fibrous encapsulation and facilitating the deposition of cite appropriate tissue ultimately yielding constructive and functional tissue remodeling.

Particularly compelling is the ability of the ceramic material to reduce or eliminate the immune response that is generated when a foreign body is implanted in a host. It is a well-recognized problem that the human immune system reacts to the presence of implant devices and the like as foreign, unnatural substances, and as a damage/danger associated molecular pattern (DAMP). Consequently, the human body responds by encapsulating it, causing bone resorption, capsular contracture and initiating a pain response. Incorporating ceramic material into the elastomer increases proliferation, differentiation and transforms growth factor beta production in normal adult human osteoblast-like cells. The hydrophilic surface of the resulting implant down-regulates pro-inflammatory cytokines interleukin 1 & 6, which modulates the immune response, facilitates wound healing, allows for early cell adhesion, and reduces pain. IL1-Beta upregulates inflammatory immune-response, and IL6-Beta has been shown to have a direct relation to pain.

Composites of ceramic material, particularly zeolite, with silicone elastomer produce a more hydrophilic and negatively charged surface which is less favorable to bacterial adhesion. The presence of the ceramic material results in a rapid transition (e.g., faster than the transition that occurs in the absence of ceramic material) from M1 proinflammatory macrophage phenotype to the M2 macrophage phenotype, thereby minimizing fibrous encapsulation, and hence likely less bacterial seeding associated with bacteremia/sepsis.

In some embodiments, either natural zeolites or synthetic zeolites may be used to make the zeolites used in embodiments disclosed herein. “Zeolite” is an aluminosilicate having a three-dimensional skeletal structure that is represented by the formula: XM_{2/n}O·Al₂O₃·YSiO₂·ZH₂O, wherein M represents an ion-exchangeable ion, generally a monovalent or divalent metal ion, n represents the atomic valency of the (metal) ion, X and Y represent coefficients of metal oxide and silica respectively, and Z represents the number of water of crystallization. Examples of such zeolites include A-type zeolites, X-type zeolites, Y-type zeolites, T-type zeolites, high-silica zeolites, sodalite, mordenite, analcite, clinoptilolite, chabazite and erionite. A-type zeolites are particularly preferred, such as 4A zeolite having particle size ranges from 1 to 10 microns with a narrow distribution of about 4 microns. Particular preferred are zeolites having an average particle size of 3-5 microns, most preferably 4 microns.

Other ceramics and metal glasses are also envisaged instead of zeolite and are within the scope of the embodiments disclosed herein. For example, zirconium phosphate, bioglass (e.g., bioactive glass particulate material or fibers, such as 45S5 (calcium sodium phosphosilicate), 58S and S70C30 bioactive glass) or silver glass could be used as the capsular contracture reducing or inhibiting agent, alone or in combination with zeolite.

In certain embodiments, when metal cation is used, the metal cation is present at a level below the ion-exchange capacity in at least a portion of the zeolite particles. In some embodiments, the amount of zeolite mixed with the polymer may range from about 5 to 50 wt. %, more preferably about 10 to 20 wt. %. The amount of metal ions, if present, in the zeolite should be sufficient such that they are present in a therapeutically effective amount when implanted into the body of a patient. For example, suitable amounts can range from about 0.1 to about 20 or 30% of the exposed zeolite (w/w %). These levels can be determined by complete extraction and determination of metal ion concentration in the extraction solution by atomic absorption or ICP OES. Preferably the ion-exchanged metal cations, if present, are present at a level less than the ion-exchange capacity of the ceramic particles. The amount of ammonium ions is preferably limited to from about 0.5 to about 15 wt. %, more preferably 1.5 to 5 wt. %. For applications where strength is not of the utmost importance the loading of zeolite can be taken as high as 50%, whether metal ion is incorporated or not. At such loadings the permeation of metal ions, where present, can permeate well below the surface layer due to interparticle contact, and much greater loadings of metal ions are possible.

In some embodiments, the ceramic material such as zeolite can be post-loaded with metal ions after it has been incorporated into the elastomer. Metal ion salt solutions, such as nitrates, acetates, benzoates, carbonates, oxides, etc., can be used to accomplish this. Addition of nitric acid to the infusion solution also may be advantageous in that it can etch the surface of the implant, providing additional surface area for ion exchange. That is, the zeolite may be charged with metal ions at a temperature between about 0 and 100° C., preferably about room temperature) from a metal ion source such as an aqueous metal ion solution, such as silver nitrate, copper nitrate and zinc nitrate, alone or in combination. Cooling to lower temperatures gives lower loading rates but higher stability. Loading at even higher temperatures can be carried out at a faster rate by maintaining the system under pressure, such as in a pressure cooker or autoclave. The content of the ions can be controlled by adjusting the concentration of each ion species (or salt) in the solution.

For example, zeolite can be loaded with metal ions by bringing the composite material into contact with an aqueous mixed solution containing ammonium ions and antimicrobial and/or therapeutic metal ions such as silver, copper, zinc, strontium, etc. These materials will strongly inhibit attachment of microorganisms and can accelerate healing and reduce inflammation and likelihood of permanent bacterial seeding of the device. By loading metal ions at these temperatures, deleterious oxidation of the metal ions that occurs at higher processing temperatures is reduced or eliminated. The most suitable temperatures at which the infusion can be carried out range from 5° C. to 75° C., but higher temperatures may also be used even above 100° C. if the reaction vessel is held under pressure. Higher temperatures will show increased infusion rates but lower temperatures may eventually produce more uniform and higher loadings. The pH of the infusion solution can range from about 2 to about 11 but is preferably from about 4 to about 7. Suitable sources of ammonium ions include ammonium nitrate, ammonium sulfate and ammonium acetate. Suitable sources of the metal ions include: a silver ion source such as silver nitrate, silver sulfate, silver perchlorate, silver acetate, diamine silver nitrate and diamine silver nitrate; a copper ion source such as copper (II) nitrate, copper sulfate, copper perchlorate, copper acetate, tetracyan copper potassium; a zinc ion source such as zinc (II) nitrate, zinc sulfate, zinc perchlorate, zinc acetate and zinc thiocyanate.

Preferably, the implant includes silicone elastomer, and ceramic particles are incorporated into the elastomer in an amount effective to mitigate or prevent capsular contracture, and/or in an amount effective such that a negative charge is imparted to an exposed surface of the elastomer. The term “exposed surface” is intended to include one or more surfaces of an implantable device that when implanted into a host, is exposed to or in contact with body tissue and/or fluid of the host.

The rate of release of therapeutic metal ions, if present, is governed by the extent of loading of the elastomer with ceramic such as zeolite and the extent to which the exposed zeolite is charged with metal ions. The electrolyte concentration in host blood and body fluids is relatively constant and will cause ion exchange with ions such as silver, copper and zinc, etc. from the surface of the implant, which deactivate or kill gram positive and gram negative organisms, including E. coli and Staphylococcus aureus. Effective antibacterial control (e.g., a six log reduction of microorganisms) is achieved even at low metal ion concentrations of 40 ppb.

Surface occupancy of the ceramic such as zeolite can be determined indirectly by post loading the ceramic with a therapeutic metal ion, removing non absorbed metal by thorough rinsing and determining the amount which can be extracted into a 1% sodium nitrate solution by ICP OES. Comparison elution from a composite without the enhanced zeolite addition to the exposed surface region will give an indication of the extent of the surface enhancement of zeolite concentration.

In some embodiments, the implant can be engineered such that a portion of the exposed surface less than the whole includes the capsular contracture reducing or inhibiting agent such as zeolite, with the remainder being naked elastomer (e.g., naked silicone elastomer) devoid of the agent.

The breast prostheses may be implanted in a host surgically. For example an incision in the breast of a host in need of a prostheses may be made and a surgical pocket formed for receiving the prosthesis. In certain embodiments, the breast prostheses may be positioned under the pectoral or pectoralis major muscle of a host e.g., submuscular pocket placement). In certain embodiments, the breast prostheses may be positioned directly behind the breast tissue, over the pectoral muscle (e.g., subglandular or subfascial pocket implantation). In certain embodiments, the breast prosthesis may be positioned partially under the pectoralis major muscle (e.g., dual plane implantation). Once the prosthesis is properly positioned, the incision may be closed.

In certain embodiments, the surgery may involve the removal of an existing prosthesis from the breast of a patient, such as an existing prosthesis that caused or is at risk to cause capsular contracture, followed by replacement with the prosthesis of embodiments disclosed herein having a reduced risk of capsular contracture.

The present disclosure is not to be limited in scope by the specific embodiments described herein. Indeed, other various embodiments of and modifications to the present disclosure, in addition to those described herein, will be apparent to those of ordinary skill in the art from the foregoing description. Thus, such other embodiments and modifications are intended to fall within the scope of the present disclosure. Furthermore, although the present disclosure has been described herein in the context of a particular implementation in a particular environment for a particular purpose, those of ordinary skill in the art will recognize that its usefulness is not limited thereto and that the present disclosure may be beneficially implemented in any number of environments for any number of purposes. Accordingly, the claims set forth below should be construed in view of the full breadth and spirit of the present disclosure as described herein.

What is claimed is:

1. A medical device configured as a breast prosthesis for implantation into a host, comprising a silicone elastomer having a capsular contracture reducing or inhibiting agent incorporated therein, said capsular contracture reducing or inhibiting agent comprising ceramic particles and being present in said elastomer in a therapeutically effective amount sufficient to inhibit capsular contracture when said device is implanted in said host.

2. The medical device of claim 1, wherein said ceramic particles comprise an aluminosilicate represented by the formula XM_{2/n}O·Al₂O₃·YSiO₂·ZH₂O wherein M represents an ion-exchangeable ion, n represents the atomic valency of the (metal) ion, X and Y represent coefficients of metal oxide and silica respectively, and Z represents the number of water of crystallization.

3. The medical device of claim 1, wherein said ceramic particles are a zeolite.

4. The medical device of claim 1, wherein said ceramic particles comprise one or more metal ions.

5. The medical device of claim 1, wherein said breast prostheses comprises an outer surface, and wherein said capsular contracture reducing or inhibiting agent is incorporated into said outer surface.

6. The medical device of claim 1, wherein said breast prostheses comprises multiple layers of said silicone elastomer, including an outer layer, and wherein said capsular contracture reducing or inhibiting agent is incorporated into at least said outer layer.

7. The medical device of claim 1, wherein said breast prostheses is implanted submuscularly.

8. The medical device of claim 1, wherein said breast prostheses is implanted subglandularly.

9. The medical device of claim 1, wherein said breast prostheses is a tissue expander.

10. A method of enhancing preferential polarization towards an M2 phenotype post-implantation of a breast prosthesis in a host, comprising implanting in said host a medical device configured as a breast prosthesis comprising a silicone elastomer having a capsular contracture reducing or inhibiting agent incorporated therein, said capsular contracture reducing or inhibiting agent comprising ceramic particles and being present in said elastomer in a therapeutically effective amount sufficient to inhibit capsular contracture when said breast prosthesis is implanted in said host.

11. The method of claim 10, wherein said ceramic particles comprise a zeolite.

12. The method of claim 10, wherein said breast prostheses is implanted submuscularly.

13. The medical device of claim 10, wherein said breast prostheses is implanted subglandularly.

14. The method of claim 10, wherein said breast prostheses is implanted during a breast reconstruction procedure.

15. The method of claim 10, wherein said breast prostheses is implanted during a breast augmentation procedure.

16. The method of claim 10, wherein said breast prosthesis is a breast implant.

17. The method of claim 10, wherein said breast prosthesis is a tissue expander.
